(19) United States
(12) Patent Application Publication
(10) Pub. No.: US 2015/0290044 A1
(43) Pub. Date: Oct. 15, 2015

(54) Bubble bandage for wound protection

(71) Applicant: Cynthia Louise Hamilton, Port Charlotte, FL (US)

(72) Inventor: Cynthia Louise Hamilton, Port Charlotte, FL (US)

(21) Appl. No.: 13/999,989

(22) Filed: Apr. 11, 2014

(51) Int. Cl. A61F 13/02

(52) U.S. Cl. CPC A61F 13/0243; A61F 13/0266; A61F 2013/00272

(57) ABSTRACT

Varying sizes of raised surfaces that are located outside an area affected by a blister, cut, wound or stitches that will absorb the pressure created from an outside force and eliminate pain from the pressure of said source.

### RELATED APPLICATIONS

The present application is a continuation-in-part application of U.S. provisional patent application Ser. No. 61/853,887, filed Apr. 15, 2013, for BLISTER BANDAID AKA BLISTER BUBBLE, by Cynthia Louise Hamilton, included by reference herein and for which benefit of the priority date is hereby claimed.

### FIELD OF THE INVENTION

The present invention relates to eliminating friction and irritation to a wound and, more particularly, to creating a raised surface on the area outside the wound which will allow pressure to be diverted away from the wound resulting in comfort for the person wearing the product.

### BACKGROUND OF THE INVENTION

Many people have had a blister, burn,cut, wound or stitches that hurt when pressure is put on the affected area. Theses blisters, burns, cuts, wounds or stitches are irritated from friction created by other bandages. Blister Bubble eliminates friction and irritation by creating a raised surface in varying sizes that are located outside the area of the blister, burn, cut, wound or stitches and thus creates an area for the pressure to absorb outside the affected area. Resulting in less pain and more comfort. This product can be used over existing bandages.

There are numerous types of blister and wound bandaids and bandages available on the market today.

None of the products available on the market today address the pain created by pressure from outside sources put on the wound.

### SUMMARY OF THE INVENTION

In accordance with the present invention, there is provided varying sizes of raised surfaces that are located outside an area affected by a blister, cut, wound or stitches that will absorb the pressure created from an outside force and eliminate pain from the pressure of said source.

It would be advantageous to provide a raised surface on the outside of a wound.

It would also be advantageous to provide a multi sizes for different sized wounds

It would further be advantageous to provide additional bandaging for further protection

### BRIEF DESCRIPTION OF THE DRAWINGS

A complete understanding of the present invention may be obtained by reference to the accompanying drawings, when considered in conjunction with the subsequent, detailed description, in which:

[symbol]

For purposes of clarity and brevity, like elements and components will bear the same designations and numbering throughout the Figures.

### DESCRIPTION OF THE PREFERRED EMBODIMENT

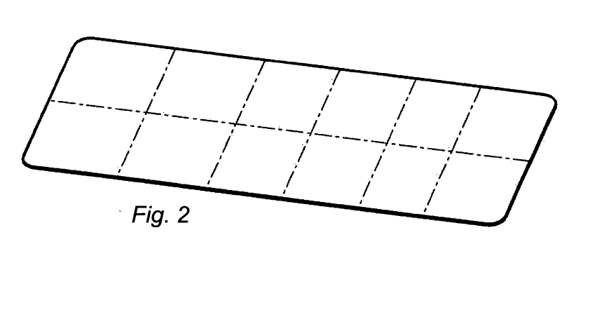

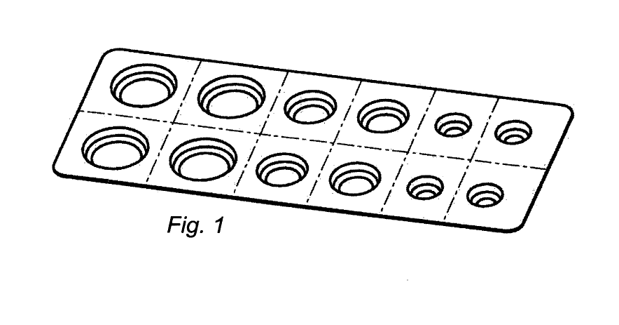

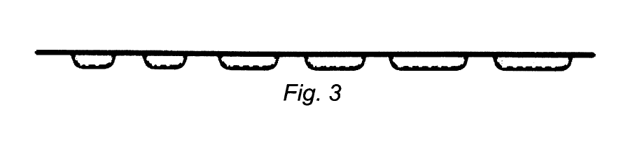

FIG. 1 is a vacuum formed,perforated and, adhesive applied blister 10

[symbol]

material is PETG with a clear color

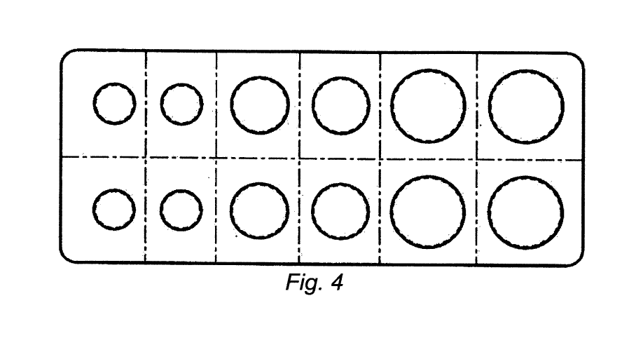

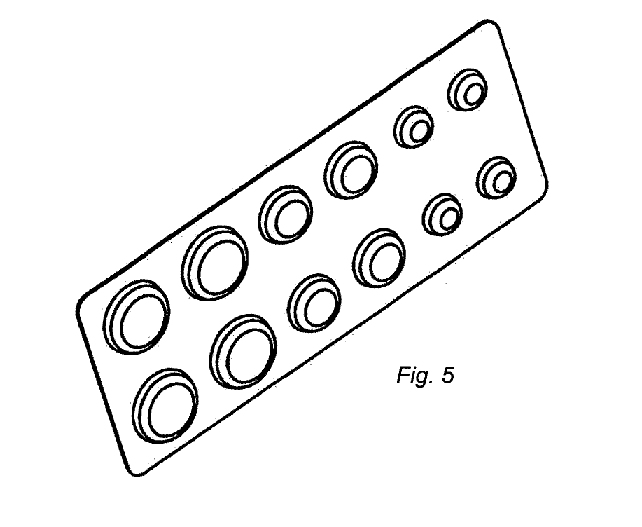

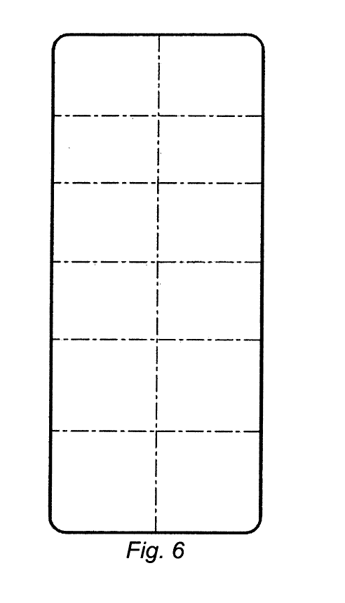

FIG. 2 is a cut and perforated a liner 12 with white color

[symbol]

Since other modifications and changes varied to fit particular operating requirements and environments will be apparent to those skilled in the art, the invention is not considered limited to the example chosen for purposes of disclosure, and covers all changes and modifications which do not constitute departures from the true spirit and scope of this invention.

Having thus described the invention, what is desired to be protected by Letters Patent is presented in the subsequently appended claims.

What is claimed is:

1. A blister bandaid aka blister bubble for eliminating friction and irritation to a wound, comprising:
means for a vacuum formed, perforated and adhesive applied blister; and
means for a cut and perforated liner.

2. The blister bandaid aka blister bubble in accordance with claim 1, wherein said means for a vacuum formed, perforated and adhesive applied blister comprises a new attribute 1 blister.

3. The blister bandaid aka blister bubble in accordance with claim 1, wherein said means for a cut and perforated liner comprises a new attribute 1 liner.

4. A blister bandaid aka blister bubble for eliminating friction and irritation to a wound, comprising:
a new attribute 1 blister, for a vacuum formed, perforated and adhesive applied blister; and
a new attribute 1 liner, for a cut and perforated liner.

5. A blister bandaid aka blister bubble for eliminating friction and irritation to a wound, comprising:
a new attribute 1 blister, for a vacuum formed, perforated and adhesive applied blister; and
a new attribute 1 liner, for a cut and perforated liner.
